(19) United States
(12) Patent Application Publication
(10) Pub. No.: US 2009/0099442 A1
(43) Pub. Date: Apr. 16, 2009

(54) TELEMETRY METHOD AND APPARATUS USING MAGNETICALLY-DRIVEN MEMS RESONANT STRUCTURE

(75) Inventors: Bradley E. Paden, Goleta, CA (US); Brian Norling, Santa Barbara, CA (US); Josiah E. Verkaik, Goleta, CA (US)

Correspondence Address: EATON PEABODY PATENT GROUP, LLC, P.O. BOX 5249, 77 Sewall Street, Suite 3000, AUGUSTA, ME, 04332-5249, US

(73) Assignee: LAUNCHPOINT TECHNOLOGIES, INC., Goleta, CA (US)

(21) Appl. No.: 12/282,593

(22) PCT Filed: Mar. 26, 2007
(86) PCT No.: PCT/US2007/064895

§ 371 Date: Sep. 11, 2008

Related U.S. Application Data
(63) Continuation of application No. 11/278,138, filed on Mar. 30, 2006.

(51) Int. Cl. A61B 3/16; A61B 5/00

(52) U.S. Cl. 600/398; 600/301

(57) ABSTRACT

A telemetry method and apparatus using pressure sensing elements remotely located from associated pick-up, and processing units for the sensing and monitoring of pressure within an environment. This includes remote pressure sensing apparatus incorporating a magnetically-driven resonator being hermetically-sealed within an encapsulating shell or diaphragm and associated new method of sensing pressure. The resonant structure of the magnetically-driven resonator is suitable for measuring quantities convertible to changes in mechanical stress or mass. The resonant structure can be integrated into pressure sensors, adsorbed mass sensors, strain sensors, and the like. The apparatus and method provide information by utilizing, or listening for, the residence frequency of the oscillating resonator. The resonant structure listening frequencies of greatest interest are those at the mechanical structure's fundamental or harmonic resonant frequency. The apparatus is operable within a wide range of environments for remote one-time, random, periodic, or continuous/on-going monitoring of a particular fluid environment. Applications include biomedical applications such as measuring intraocular pressure, blood pressure, and intracranial pressure sensing.

### FIELD OF THE INVENTION

The present invention relates generally to an apparatus including a resonant structure suitable for measuring quantities convertible to mechanical stress or mass in the resonant structure and a related method. More particularly, the present invention relates to an apparatus and method including a magnetically-driven resonant sensor suitable for wireless physiological parameter measurement and telemetry within a living body.

### BACKGROUND OF THE INVENTION

Within the field of biomedical devices, the measurement of physiological parameters within a living body presents unique problems. Such problems and related known solutions can be found, for example, in the treatment of glaucoma which is a highly significant concern to the medical community. Glaucoma is a serious disease that can cause optic nerve damage and blindness. There are a number of causes of glaucoma, but increased intraocular pressure is the primary mechanism. Because of the large number of persons suffering from glaucoma combined with the seriousness of the disease and the need for early detection and optimized drug treatment, it is desirable to obtain frequent measurements of eye pressure. Moreover, eye pressure can vary throughout the day such that clinical diagnosis, based on infrequent testing, is often delayed. It is therefore desirable to obtain fast and accurate pressure monitoring.

The surgical placement of a sensor in the eye (i.e., intraocular) may be advisable in patients with glaucoma or in patients with a risk of glaucoma if they are undergoing eye surgery for another reason. In particular, patients receiving an intraocular lens (IOL) can be fitted with pressure sensors attached to the IOL with little additional health risk or cost. Also, glaucoma patients who need to adjust their drug dosage according to eye pressure would benefit from such a device.

There have been a number of past devices directed at the measurement of intraocular pressure. A prevalent technique exists that employs contacting the cornea of the eye using a tonometer. The cornea is topically anesthetized and brought into contact with the smooth, flat surface of the tonometer probe. The amount of pressure required to flatten a specified area of the cornea is used to compute the intraocular pressure. While this method is cost effective, it suffers from a number of significant drawbacks. For example, a trained clinician is required for the measurement so that frequent monitoring is not possible. Further, the mechanical properties of the cornea can affect the measurement. Still further, the tonometer needs to be maintained in clean and sterile conditions.

It has elsewhere previously been proposed to provide a technique for continuously monitoring eye pressure involving an inductor-capacitor (LC) resonant circuit wherein the resonant frequency was sensitive to eye pressure. However, such devices were not sufficiently compact and reliable for clinical use in humans, and lacked a method of implantation and attachment. Moreover, LC resonant sensors fail to provide a sufficiently sharp resonance to allow for rapid and simple external sensing of frequency and hence pressure. Such sensors may exhibit a quality factor (Q) in the range of 30. The Q factor is a measure of the “quality” of a resonant device or system. Resonant systems respond to frequencies close to their natural frequency much more strongly than they respond to other frequencies. The Q factor indicates the susceptibility to resonance in a system. Systems with a high Q factor resonate with greater amplitude (at the resonant frequency) than systems with a low Q factor. Damping decreases the Q factor. Modifications to known LC resonators using planar microelectromechanical systems (MEMS) manufacturing technologies have been attempted. However, the problems of low Q associated with resistive losses in the coil and other conductors remained due to sensitivity of such system to the relative position of the sensor and the inductive pick-up coil.

While still other pressure sensors derived from a mechanical resonator have been suggested that could be small enough for implantation in the eye and still have a high Q, such sensors often use light to drive a photo-diode that electrostatically attracts a resonant beam or otherwise provides an optical excitation system delivering the requisite high light intensities to the sensor. The relatively high intensity light requirements may interfere with the patient's vision or may otherwise not likely be suitable for use near the human eye.

There also exist a number of LC resonant pressure sensors with wireless communication. Such schemes rely on magnetic coupling between an inductor coil associated with the implanted device and a separate, external “readout” coil. For example, one known mechanism of wireless communication is that of the LC tank resonator. In such a device, a series-parallel connection of a capacitor and inductor has a specific resonant frequency that can be detected from the impedance of the circuit. If one element of the inductor-capacitor pair varies with some physical parameter (e.g., pressure), while the other element remains at a known value, the physical parameter may be determined from the resonant frequency. Such devices using LC resonant circuits have been proposed in various forms for many applications such as hydrocephalus applications, implantable devices for measuring blood pressure, and implantable lens for monitoring intraocular pressure.

Implantable wireless sensors have also existed within the treatment of cardiovascular diseases such as chronic heart failure (CHF). CHF can be greatly improved through continuous and/or intermittent monitoring of various pressures and/or flows in the heart and associated vasculature. While applications for wireless sensors located in a stent have been suggested, no solution exists to the difficulty in fabricating a pressure sensor with telemetry means sufficiently small enough for incorporation into a stent.

In nearly all of the aforementioned cases, the disclosed devices require a complex electromechanical assembly with many dissimilar materials. This typically results in significant temperature and aging-induced drift over time. Such assemblies may also be too large for many desirable applications—e.g., including intraocular pressure monitoring and/or pediatric applications. Finally, complex assembly processes make such devices prohibitively expensive to manufacture for widespread use. Such manufacturing complexity only increases with alternative process that form microfabricated sensors which have recently been proposed as an alternative to conventionally fabricated devices.

There have also been attempts to offer telemetry sensors using magneto-mechanical pressure sensors of the magnetostrictive type. Magnetostriction is a property of a ferromagnetic material that changes volume when subjected to a magnetic field. When biased by a non-alternating magnetic field, magnetostrictive material stores energy via mechanical strain. This storage affects the Young's modulus, E, of the material. Such magnetostrictive materials can be caused to resonate in an alternating magnetic field. Resonant frequency can be designed by varying the geometry of the material, one or more mechanical properties of the magnetostrictive material, and strength of the biasing non-alternating magnetic field. These types of sensors have a high magnetic permeability element. The high magnetic permeability element is placed adjacent to an element of higher magnetic coercivity. The high magnetic permeability element being adjacent to the element of higher magnetic coercivity resonates when interrogated by an alternating electromagnetic field due to nonlinear magnetic properties. The high magnetic permeability element adjacent to the element of higher magnetic coercivity generates harmonics of the interrogating frequency that are detected by a receiving coil. Such sensors can have a thin strip of magnetostrictive ferromagnetic material placed adjacent to a magnetic element of higher coercivity (often referred to as “a magnetically hard element”).

As suggested above, the non-alternating magnetic bias placed on the magnetostrictive material causes a mechanical strain in the magnetostrictive material that in turn affects a resonant frequency of the magnetostrictive material. The resonance of the magnetostrictive material can be detected electromagnetically. While magneto-mechanical pressure sensors have advantages such as high operating reliability and low manufacturing cost over previous electromagnetic markers of high sensitivity, there are known problems associated with such a pressure sensor. The magnetostrictive response is temperature sensitive, primarily due to a dependence on Young's modulus. Consequently, such magnetostrictive pressure sensors often require independent temperature correction that involves the use of additional temperature and measurement devices that add size and preclude construction as a single monolithic structure or adaptation to a micro-miniature size suitable for monitoring physiological parameters.

Further known types of mechanical resonant sensors have been used for many years to achieve high accuracy measurements. Vibrating transducers have been used in accelerometers, pressure transducers, mass flow sensors, temperature and humidity sensors, air density sensors, and scales. Such sensors operate on the principle that the natural frequency of vibration (i.e., resonant frequency of an oscillating beam or other member) is a function of the induced strain along the member. One of the primary advantages of resonant sensors is that the resonant frequency depends only on the geometrical and mechanical properties of the oscillating beam, and is virtually independent of electrical properties. As a result, precise values (e.g., resistance and capacitance) of drive and sense electrodes are not critical. A possible disadvantage is that any parasitic coupling between the drive and sense electrodes may diminish accuracy of the resonant gauge. Furthermore, in a conventional capacitive drive arrangement, the force between the oscillating beam and drive electrode is quadratic, resulting in an unwanted frequency pulling effect. While crystalline quartz piezoresistors have been satisfactorily employed in resonant gauge applications, their size limits their practical utility.

Recently, other known types of pressure sensing devices have been fabricated from semiconductor material—e.g., silicon. In general, pressure sensing devices of this type are realized adopting so-called “silicon micromachining” technologies. Such technologies provide two or three-dimensional semiconductor structures with mechanical properties that can be well defined during design, despite their extremely small size (down to a few tens of microns). Accordingly, such semiconductor structures are capable of measuring and/or transducing a mechanical quantity (for example the pressure of a fluid) with high accuracy, while maintaining the advantages, in terms of repeatability and reliability that are typical of integrated circuits. Such pressure sensing devices made of semiconductor materials of the so-called “resonant-type” pressure sensing devices have become widespread in the industrial field. Ultra miniaturized sensors for minimally invasive use have become important tools in heart surgery and medical diagnoses during the last ten years. Typically, optical or piezoresistive principles have been employed in such sensors. Although these devices have considerable advantages, such as, for example, high accuracy and stability of measurement even for very wide measurement ranges (up to several hundred bars), such known sensors suffer from some drawbacks. In particular, calibration is fairly complicated and manufacture is not an easy task, producing fairly high rejection rates of the finished products. Accordingly, there is much unresolved need for new types of sensors and other means and methods of making ultra miniaturized sensors in an efficient and economic way.

There are also known related devices pertaining to magnetically driven cantilevers for use in atomic force microscopes and imaging processes involving magnetic force microscopy. Still further, there are known related devices pertaining to micro-compasses with magnetically coupled resonant structures. However, such cantilevers and micro-compasses fail to provide a solution in measuring other quantities convertible to measuring changes in mechanical stress (i.e., pressure and force).

In view of the above and other limitations on the prior art, it is apparent that there exists a need for an improved sensor system. It is, therefore, desirable to provide a wireless MEMS system utilizing a magnetically-driven resonator for use in physiological parameter measurement capable of overcoming the limitations of the prior art and optimized for signal fidelity, transmission distance, and manufacturability. It is further desirable to provide a magnetically-driven MEMS resonator adapted for wireless physiological parameter measurement including resonant structure attached to magnetic material used to drive structure resonance.

### SUMMARY OF THE INVENTION

In general, the present invention relates to telemetry using sensing elements remotely located from associated pick-up, and processing units for the sensing and monitoring of pressure within an environment. More particularly, the invention relates to a unique remote pressure sensing apparatus that incorporates a magnetically-driven resonator (whether hermetically-sealed within an encapsulating shell or diaphragm) and associated new method of sensing pressure. The resonant structure is suitable for measuring quantities convertible to changes in mechanical stress or mass. This structure can, for example, be integrated into pressure sensors, adsorbed mass sensors, and strain sensors. The present invention includes a magnetically-coupled MEMS resonator that provides improvements over known devices including increased reliability and ease-of-use.

The pressure sensing apparatus and method(s) in accordance with the present invention provide information by utilizing, or listening for, the resonant frequency of the oscillating resonator. The resonant structure listening frequencies of greatest interest are those at the mechanical structure's fundamental or harmonic resonant frequency. The pressure sensing apparatus of the invention can operate within a wide range of environments for remote one-time, random, periodic, or continuous/on-going monitoring of a particular fluid environment.

Any of a number of applications for the present apparatus and method is contemplated including, without limitation, biomedical applications (whether in vivo or in vitro). The resonant structure in accordance with the present invention is driven and sensed remotely, allowing use in applications where connection by way of wires is impractical or not otherwise feasible. In particular, the present apparatus and method is suitable for biomedical applications including measuring intraocular pressure in patients with glaucoma or patients at risk for contracting glaucoma and having intraocular lenses (IOL's). While this specific application relating to glaucoma and measurement of intraocular pressure is discussed in detail, it should be understood that such specific example is merely illustrative of the present invention and other biomedical applications with the same limitations as the intraocular environment may equally benefit from the present invention such as, but not limited to, blood pressure sensing and intracranial pressure sensing. Moreover, the present invention may be useful in applications pertaining to rotating machinery, not limited to biomedical applications, as another specialized application where wires are often impractical.

Energy is transmitted to the resonant structure magnetically and the motion of the structure is detected magnetically, optically, or acoustically. Magnetic drive is particularly useful because of the ability to provide high forces with the magnetic drive coils separated by a sizable distance. The sensing apparatus of the present invention is useful to measure intraocular pressure, but can be applied to any sensing application where the sensed variable can affect a change in stress or mass in a mechanical resonator so that its frequency is altered. In the case of intraocular pressure, structure motion may be detected magnetically or optically.

In one embodiment of the invention, a magnetic material is mounted on a torsional resonator. Pressure is converted to tension in the resonator beams so that its frequency is correlated to pressure. The torsional resonator is excited by a nearby current carrying coil and the same coil can be used for sensing the resonant frequency. The coil is connected to a grid dip meter or other circuit to enable the measurement of the resonance. The sensor may be hermitically sealed in a miniature capsule and attached to an IOL implanted in the eye. Alternatively, it can be attached directly to the iris. A variation on this embodiment replaces the permanent magnet with a soft magnetic material such as nickel-iron, cobalt-iron or other alloy that can be easily attached or formed onto the resonator. During use, soft magnetic material is magnetized with a permanent magnet external to the eye. The resonator is excited with a coil as mentioned above.

An advantage of the present invention is the high quality factor (Q) that is attainable with mechanical resonant structures relative to LC resonant circuits and the improved reliability and ease-of-use of a sensor based on a high-Q resonator. Further, magnetic couplings allow for communication with the sensor through biological tissues. The resonant structure includes a magnetic material and is adapted to vibrate in response to a time-varying magnetic field. The apparatus also includes a receiver to measure a plurality of successive values magnetic field emission of the vibrating structure taken over an operating range of successive interrogation frequencies to identify a resonant frequency value for said sensor.

Another aspect of the present invention is to provide a pressure sensing apparatus for operative arrangement within an environment that incorporates a resonant structure with at least one magnetically-driven resonant beam that will vibrate in response to a time-varying magnetic field (whether radiated continuously over an interval of time or transmitted as a pulse). The resonant beam may be enclosed within a hermetically-sealed diaphragm, at least one side of the diaphragm having a flexible membrane to which the resonant structure is coupled. The pressure sensing apparatus also includes a receiver unit capable of picking up emissions (whether electromagnetic or acoustic) from the sensor. Preferably, the receiver (a) measures a plurality of successive responses corresponding to the frequency of the sensor taken over an operating range of successive interrogation frequencies to identify a resonant frequency value for the sensor, or (b) detects a transitory time-response of resonance intensity of the sensor due to a time-varying magnetic field pulse to identify a resonant frequency value thereof. In the latter case, the detection can be done after a threshold amplitude value for the transitory time-response of residence intensity has been observed; then a Fourier transform can be performed on the transitory time-response of the emission to convert the detected time-response information into the frequency domain.

It is an aspect of the present invention to provide a sensing apparatus for measuring quantities convertible from changes in physical observations, the apparatus including: a resonant structure responsive to the changes in the physical observations, the resonant structure including a magnetized element; an electromagnetic coil operationally coupled to the magnetized element, the electromagnetic coil being an excitation coil magnetically coupled to the magnetized element to excite a resonance of the resonant structure; and, a signal processor for processing movement of the resonant structure, the signal processor correlating the movement with regard to the changes in the physical observations so as to produce sensed data. The resonant structure includes: a substrate locatable in an environment to be monitored, a flexible diaphragm hermetically sealed to the substrate and in communication with the environment to be monitored, a sealed chamber encompassed by the substrate and at least one flexible diaphragm, and a resonant beam connected to the magnetized element, the resonant beam suspended within the sealed chamber and mechanically coupled to the flexible diaphragm, wherein the magnetized element oscillates the resonant beam in response to an electromagnetic signal generated by the signal processor and formed by the electromagnetic coil.

It is another aspect of the present invention to provide a method of sensing physical observations within an environment, the method including: operatively arranging a resonant structure in the environment and in proximity to a direct current bias field, the resonant structure including a magnetized element and being responsive to changes in the physical observations; applying a magnetic field by way of an electromagnetic coil operationally coupled to the magnetized element; measuring a plurality of successive values for magnetic resonance intensity of the resonant structure with a signal processor operating over a range of successive interrogation frequencies to identify a resonant frequency value of the resonant structure; and using the resonant frequency value to identify sensed data correlating to the physical observation of the environment.

Many advantages exist by providing the flexible new pressure sensing apparatus of the invention and associated new method of sensing pressure of an environment using a sensor with at least one magnetically-driven resonant structure. Such advantages include, but are not limited to, the following:

(a) Sensitivity—The method provides a means for achieving high sensitivity and high-Q resonance frequency.

(b) Simplicity—Resonance frequency is easily measure, and the small devices can be manufactured in arrays having desired acoustic response characteristics.

(c) Speed—Much faster response time (tens of microseconds) than conventional acoustic detectors (tens of milliseconds) due to extremely small size and large Q value.

(d) Variable Sensitivity—The sensitivity can be controlled by the geometry of the microbeam(s) and the coating thereon. This can be made very broadband, narrow band, low pass, or high pass.

(e) Size—Current state-of-the-art in micro-manufacturing technologies suggest that a mechanical structure could be mounted on a monolithic MEMS structure.

(b) Low power consumption—The power requirements are estimated to be in sub-milliwatt range for individual sensors.

(d) Low cost—No exotic or expensive materials or components are needed for sensor fabrication. Electronics for operation and control are of conventional design, and are relatively simple and inexpensive.

(e) The invention can be used for one-time, periodic, or random operation, or used for continuous on-going monitoring of pressure changes in a wide variety of environments; Sensor materials and size can be chosen to make one-time, disposable use economically feasible.

(f) Versatility—The invention can be used for operation within a wide range of testing environments such as biomedical applications (whether in vivo or in vitro).

(g) Simplicity of use—The new sensor structure can be installed/positioned and removed with relative ease and without substantial disruption of a test sample or environment.

(h) Structural design flexibility—the resonant structure may be formed into many different shapes and may be fabricated as a micro-circuit for use where space is limited and/or the tiny sensor must be positioned further into the interior of a sample or environment being tested/monitored.

(i) Several sensors may be positioned, each at a different location within a large test environment, to monitor pressure of the different locations, simultaneously or sequentially.

(j) Several sensor elements may be incorporated into an array to provide a package of sensing information about an environment, including pressure and temperature changes.

(k) Receiving unit design flexibility—One unit may be built with the capacity to receive acoustic emissions (elastic nonelectromagnetic waves that can have a frequency up into the gigahertz (GHz) range) as well as frequency of the resonant structure, or separate acoustic wave and electromagnetic wave receiving units may be used.

Other advantages and benefits may be possible, and it is not necessary to achieve all or any of these benefits or advantages in order to practice the invention. Therefore, nothing in the forgoing description of the possible or exemplary advantages and benefits can or should be taken as limiting.

### BRIEF DESCRIPTION OF THE DRAWINGS

The novel features of the present invention, which are considered as characteristic for the invention, are set forth in this disclosure, but not with particularity according to limiting claims. The invention itself, however, both as to organization and methods of operation, together with further objects and advantages thereof, may best be understood by reference to the following description, taken in conjunction with the accompanying drawings in which:

FIGS. 1a and 1b show top and side views, respectively, of a basic resonator structure with attached permanent magnet.

FIG. 2a shows a coil and resonator structure.

FIGS. 2b-2d show three of the many modes of vibration of the resonator illustrated in FIG. 2a.

FIGS. 3a and 3b show an embodiment of the resonator structure with a soft magnetic material.

FIGS. 4a and 4b show a dynamically balanced embodiment with minimal base motion.

FIG. 5 shows an alternative embodiment with two magnets on the same beam.

FIG. 6 shows an embodiment with additional flexures to allow alignment with a large external DC field;

FIG. 7 shows a resonant structure incorporated into a pressure sensor.

FIG. 8 shows an embodiment of an adsorption-type chemical sensor.

FIG. 9 shows a pressure sensor incorporated into an intraocular lens.

FIGS. 10a and 10b show coil placements outside of an eye.

FIG. 11 shows transmit and receive signals to/from the coil.

FIG. 12 illustrates the signal structure.

FIG. 13a shows the signal processor of the present invention.

FIG. 13b shows the signal processor used with an LC type sensor.

FIGS. 14a and 14b show software functions for the receiving signal.

### DETAILED DESCRIPTION

Generally, the present invention provides a method and apparatus including a magnetically-driven resonant structure suitable for measuring some change in a physical observation—e.g., sensing change in pressure, flow, etc. However, for purposes of illustration, the present invention is discussed in terms of a method and apparatus suitable for measuring intraocular pressure in patients having glaucoma or patients at risk of contracting the disease and having intraocular lenses (IOL's). As discussed earlier, previous devices fail to meet dimensional requirements, or they suffer from sensitivity limitations needed for wireless physiologic parameter measurement within a living body.

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways without straying from the intended scope of the present invention. Furthermore, unless otherwise indicated, the terms and expressions employed herein have been chosen for the purpose of describing the illustrative embodiments of the present invention for the convenience of the reader and are not for the purpose of limiting the invention. Further, it is understood that any one or more of the following-described embodiments, expressions of embodiments, examples, etc., can be combined with any one or more of the other following—described embodiments, expressions or embodiments, examples, etc.

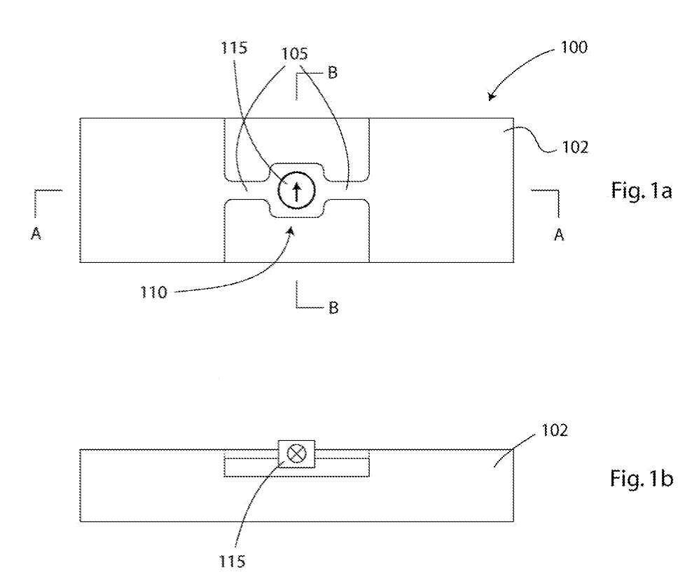

FIGS. 1a and 1b depict a simple embodiment of the invention. FIG. 1a is a top view and FIG. 1b is a section view along section A-A. In reference FIGS. 1a and 1b, a resonant structure 100 includes a body 102, elastic beams 105, a mass 110 and a magnetic material 115 mounted on the mass 110. The beam materials in particular are chosen such that they have relatively low damping and the mass can sustain a vibrational motion if excited. Typically, the body 102, elastic beams 105, and mass 110 are fabricated from the same elastic material. Suitable materials are single crystal silicon, polycrystalline silicon, titanium, brass or any other elastic material with low damping. As with many elastic systems, the resonant structure 100 can vibrate in a number of vibrational modes. As is done in the art, mode shapes and modal frequencies are associated with each vibrational mode.

Three such mode shapes are depicted in FIG. 1c. Mode shape 120 represents an up and down motion relative to the equilibrium position 135. At one extreme, the mass and elastic beams deflect upward to the mode shape 120. At the other extreme, the mass 110 and elastic beams 105 deflects downward to the mirror image of 120 relative to 135. Mode shape 125 represents a second vibrational motion of the mass 110 and beams 105 wherein the mass rotates back and forth about an axis pointing out of FIG. 1c. Another mode shape is associated with the motion 130 depicted in FIG. 1d.

In general, a resonant structure is any material body that vibrates at one or more frequencies. Examples include: stringed musical instruments, tuning forks, chimes, quartz crystals in watches, and microelectromechanical systems (MEMS) with vibrating components such as MEMS vibrational gyros. In the case of a guitar, the frequencies of vibrations include those of the strings, including their harmonic motions.

An advantage of the embodiment shown in FIGS. 1a through 1c is simplicity. However, vibrations of the beams and mass are accompanied by vibrations of the body. Consequently, if the body is brought into contact with a support structure, vibrational energy is drawn from the resonant structure and the vibration decays away more quickly than in resonant structures where the support locations vibrate little or not at all. The rate of decay of a vibration is captured in the notion of a quality factor (Q) by those practicing the art of vibration analysis. Higher quality factors reflect more sustained vibrations and can be as high as 1,000,000 in some single crystal resonant structure made from quartz or silicon.

In reference to FIG. 1c, forces F and/or moments M transmit stresses to the resonator structure and tension to the beams 105 in particular. Such stresses change the modal frequencies. Such a system is an example of a frequency variable resonator dependent on force. Force is an example of a sensed quantity and the embodiment of FIG. 1c can function as a force sensor. Mode shape 130 has a modal frequency that is relatively independent of beam tension when the beams are cylindrical rods. Hence, the cross section and choice of mode must be optimized to obtain the best sensitivity. This is easily done with commercial finite-element analysis (FEA) software packages such as COSMOS™ or ANSYS™. Because many sensed quantities such as pressure, strain, acceleration, and chemical concentration can be converted to stress in the resonant structure, the embodiment of FIGS. 1a through 1c can be incorporated into various sensors. Further, the rotation of the body can cause amplitude variations and energy transfer between modes. Such a phenomenon can be used to design a vibrational gyro. In this later case, we say that the resonator is an amplitude variable resonator dependent on rotation. Rotation is another example of a sensed quantity.

The magnetic material 115 in FIG. 1a provides a mechanism to excite the vibration in the resonant structure by coupling externally applied magnetic fields to the magnet. Vibrations are particularly excited when the external magnetic field applies oscillatory forces and/or torques to the magnetic material at the modal frequencies. The coupling is further enhanced when the mode shape is such that the magnet translates or rotates significantly when the mode is excited. For example, mode shapes 120, 125, and 130 all rotate or translate the magnetic material. The magnetic material may be a magnetized hard magnetic material (i.e., a permanent magnet such as NdFeB, SmCo or Ferrite) or a soft magnetic material such as silicon-iron or cobalt-iron. When a soft magnetic material is used, it is preferable to magnetize the soft material with a DC field produced by an external permanent magnet or a DC current in a coil.

Relationships can be computed for the force/torque interactions between a magnetic material and a magnetic field, and the interaction between these forces/torques and the motion of a resonant structure. If geometries are simple, pencil and paper calculations can be used. More complex geometries can be analyzed with finite-element software. In this way, the entire system can be engineered and optimized prior to fabrication and testing.

Detection of motion in the invention of FIGS. 1a through 1c can be accomplished magnetically through, for example: the use of a pick-up coil; acoustically by detecting vibrations of the body directly or via a propagating medium; or optically by reflecting light (e.g., laser light) off a polished surface of the structure.

The fabrication of the embodiment of FIGS. 1a through 1c can be accomplished with a number of manufacturing methods. When the device is small, MEMS manufacturing methods using silicon are desired. These methods include photolithography, etching (e.g., anisotropic etching, isotropic etching, and deep reactive ion etching), and various bonding techniques. Unique to the present invention is the bonding of the magnetic material 115 to a resonant structure 100. If a hard (i.e., high coercivity) magnetic material such as NdFeB or SmCo is used, the magnetic material is preferably bonded to the remaining structure with epoxy, photoresist, or other suitable organic compound. Another method of attaching materials such as NdFeB is to electroplate the NdFeB surface with nickel and then gold. The gold can then be bonded to silicon thermally though eutectic bonding. Alternatively, if a soft magnetic material is attached, electroplating using methods developed for disk drive recording heads are preferred.

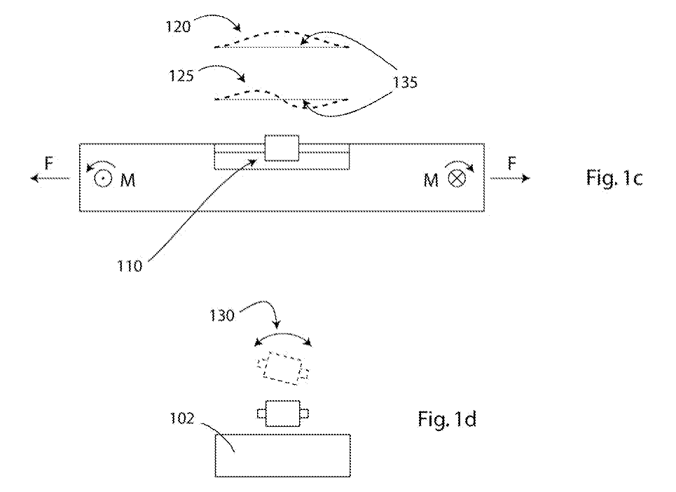

FIGS. 2a through 2d depict configurations for exciting and/or detecting vibrations when a permanent magnet (PM) is attached to the resonant structure in various orientations. The magnetization direction 215 is shown. FIG. 2a depicts a simple coil 200 with terminals 205 and 210 formed of insulated copper wire or another such suitable electrical conductor. To excite motion about the axis 220 in the resonant structure, electrical current is passed through such a coil 200 in order to produce a magnetic field. If the current waveform contains a frequency component at a resonant frequency, the corresponding vibrational mode can be excited. The orientation of the coil 200 relative to the PM direction of magnetization is important. For maximal torque application to the PM, the applied magnetic field should be perpendicular to the direction of PM magnetization. For maximal force application to the PM, the applied magnetic field gradient should be aligned with the direction of PM magnetization. In general, there will be a combination of torques and forces on the PM due to the combined effects of the magnetic field and the magnetic field gradient. Other angles differing from these can work well, but angles that differ from these by exactly 90 degrees produce no torque or force respectively.

The coil 200 can also sense rotary and linear motion of the PM as these motions generate a voltage across the coil terminals. Fortuitously, the relative position and orientation of the coil 200 and PM that maximize torque and force also maximize the voltage generated due to rotary and linear motion, respectively. While the application of a current while the sensing of voltage is one way to measure the resonant frequency of the resonant structure, one could also apply a voltage to the coil 200 while measuring the current. It should be noted that the positioning of magnetic material in a resonant structure near a coil or collection of coils alters the electrical properties of the coil(s). In particular, resonant frequencies can be measured. These changes in electrical properties of the coil(s) can be measured with signal processing devices which implement signal processing functions in analog circuits, digital circuits, and/or software controlled circuits. In particular, one or more of the resonant frequencies of the structure can be determined in this way. For example, the impedance of a single coil (such as 200 shown) will drop near a resonance of the structure incorporating a PM. An impedance analyzer or grid dip meter can serve to measure the changes in electrical properties of the coil. Also, the resonant structure/permanent magnet/coil system can be used to set the frequency of an electrical oscillator, as does a quartz crystal. Other signal processing devices are described below.

FIG. 2b depicts a mechanism for exciting motion along the directions 225. Other such mechanisms for exciting motion along 230 and about the axis 220 are shown in FIGS. 2c and 2d respectively.

FIG. 2d, in addition to depicting a possible motion of the resonator, depict the use of soft magnetic material 235 exterior to the resonator to improve the magnetic coupling between the coil and the resonator.

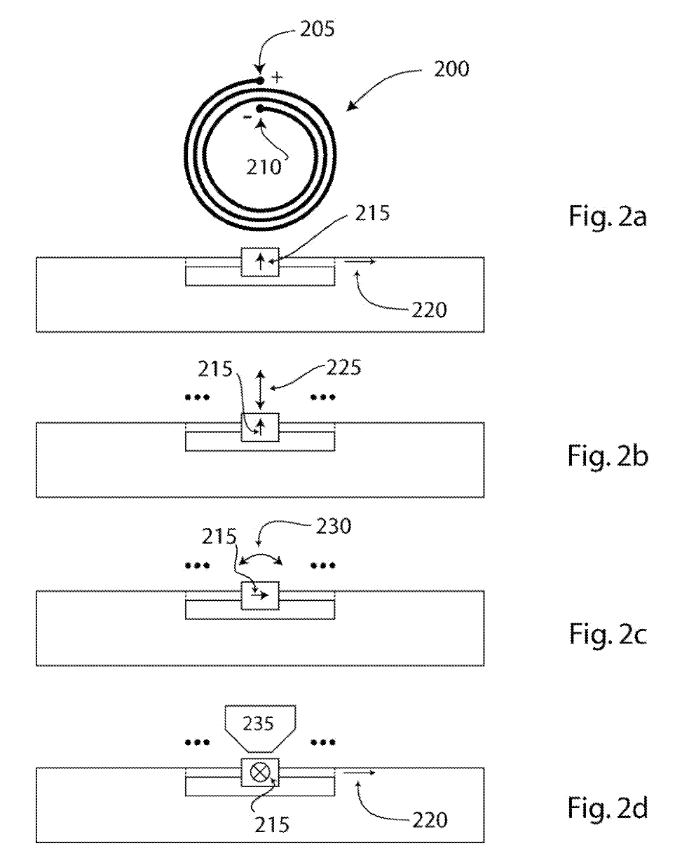

FIG. 3a depicts a system employing a soft magnetic material 300 wherein the magnetization arrow 305 is induced by an external magnetic field. FIG. 3b depicts a section of the same embodiment along cross section C-C. Further, FIG. 3b depicts a permanent magnet 310 magnetized out of the page at location 315 and producing a magnetic field into the page at locations 320 and others. In particular, the permanent magnet produces a magnetizing field for the soft magnetic material that magnetizes the material into the page in FIG. 3b and along the direction 305 in FIG. 3a. Once this soft material is magnetized, it can be excited by an AC current in a coil 325 in a fashion similar to those noted in FIGS. 2a through 2d.

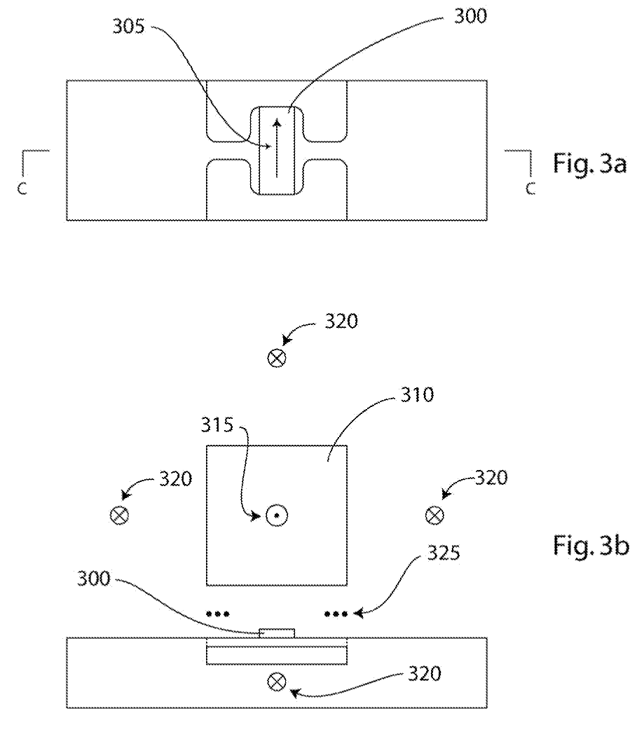

FIG. 4a depicts another embodiment of the invention wherein the mode shape of interest is symmetric, as shown in FIG. 4b which is taken across line D-D. The symmetry allows the vibration to occur with insignificant motion of the body 402. Thus, little energy is transferred to any structure supporting the body and the mode of interest will have a high Q because the losses to the surrounding structure are minimized. By analogy, a similar design principle is applied to musical tuning forks. A tuning force vibrates in a desired mode shape, but the handle of the fork does not, so tuning forks have a relatively high Q. A double-ended tuning fork (DETF) is a commonly used resonator structure and represents another resonator embodiment useful in our invention. The essential feature of these mode shapes is the insignificant motion of the supported body or supported points—this feature is referred to as dynamic balance. Geometric symmetry is common for a system with dynamic balance, but it is not essential. For example, the embodiment of FIG. 4a needs only one magnet and dynamic balance can be accomplished with an equivalent mass instead of the magnet. However, the embodiment of FIG. 4a employs opposing permanent magnet magnetizations including masses 455 and beams 405. The net dipole moment is nearly zero so that the system is not subjected to torque in an ambient magnetic field. This is beneficial if the sensor is to be used in magnetic medical imaging equipment (e.g., magnetic resonance imaging (MRI)) provided that the magnets are not demagnetized.

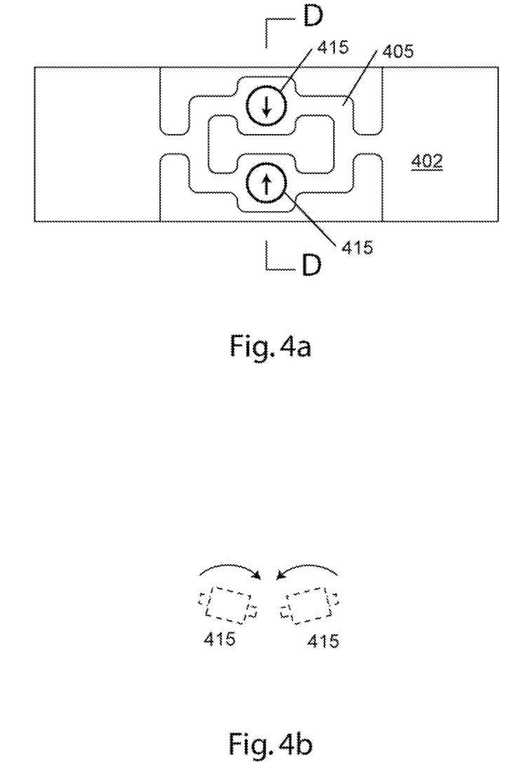

FIG. 5 is another embodiment shown in a snapshot during vibration. This design also has no net magnetic moment. It has multiple magnets 515 on a single beam and incorporates mechanical amplification of forces F and 2F. The mechanical amplification is accomplished in this elastic system through lever arms 500. In a force sensor, mechanical amplification converts (i.e., “focuses”) a higher fraction of the mechanical energy transmitted to the resonator by the external forces into mechanical strain energy in the resonant structure. This is done to maximize the frequency shift in the mode of interest. Here, the term mechanical amplification is used to mean this kind of focusing of mechanical energy.

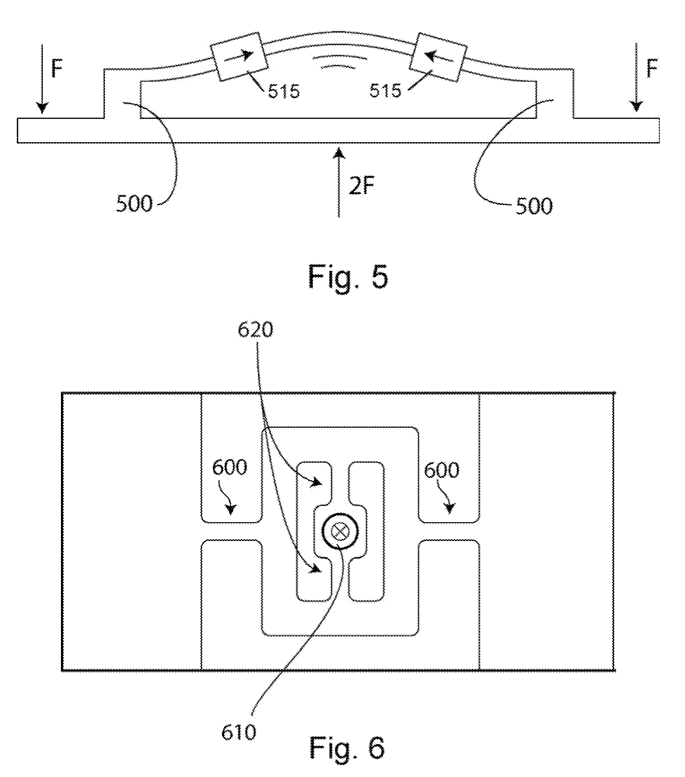

FIG. 6 depicts an embodiment with an additional set of flexible beams 600 and 620, permanent magnet 610 and surrounding mass. The beams 620 are intended to undergo the largest vibrational motion. The beams 600 allow additional rotation of the permanent magnet so that the magnet can align with a large external magnetic field due to, for example, an MRI. In this way, torque transmitted to the body of the resonant structure can be reduced. In turn, when used in the human body, torque to supporting tissues is reduced.

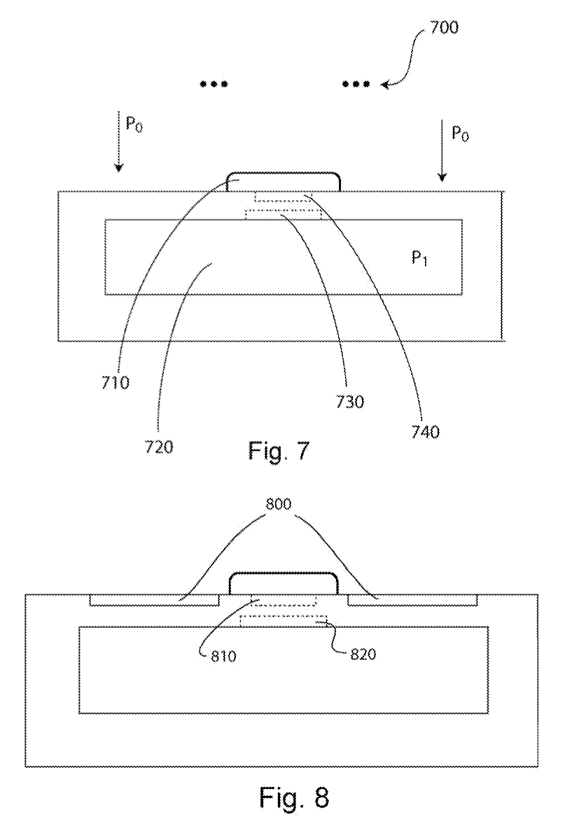

FIG. 7 depicts both a pressure sensor including a coil 700, sealed volumes 710 and 720 and two resonant structures 730 and 740 used in a differential mode. The embodiment includes sealed volumes to protect the resonant structures and create a reference pressure in volume 720. Resonator 740 is subjected to compressive loading when a pressure P0>P1 is applied and resonator 730 (operating in a different frequency range) is subjected to tensile loading. By knowing the temperature sensitivity of the frequencies of the resonant structures in this system, one can solve for the pressure difference P0−P1 independent of temperature. This is called a differential sensor. An exact or weighted difference of the frequency shifts might be used. In general, a weighted difference can be optimized to give the best rejection of temperature effects. Gas expansion effects when P1 is not zero (i.e., a vacuum) can also be accommodated in calculations. Further, more than two sensors can be used in differential mode. The frequency outputs of M resonant structures can be used to solve for M different quantities provided that the M equations relating the measured quantities to the frequency are not singular. Even if just one quantity is of interest, multiple sensors improve the estimate of that quantity. The volume of the sealed volumes 710 and 720 may be chosen to be relatively large so that a small amount of out-gassing from the materials would have an insignificant effect on the reference pressure.

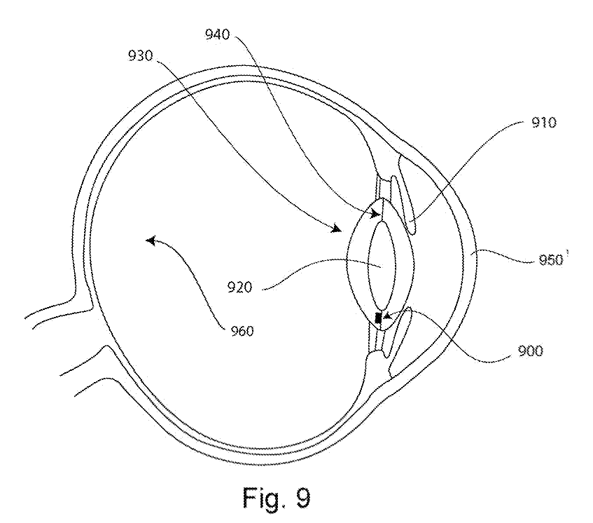

FIG. 8 shows a modification of the pressure sensor of FIG. 7 to form a chemical sensor. Material 800 that preferentially adsorbs a chemical(s) of interest is incorporated into the sensor. If the chemical(s) are present, they are adsorbed and change the mechanical stress levels in the adsorbent material. This stress is transmitted to the resonant structures 810 and 820 and causes a shift in their resonant frequencies.

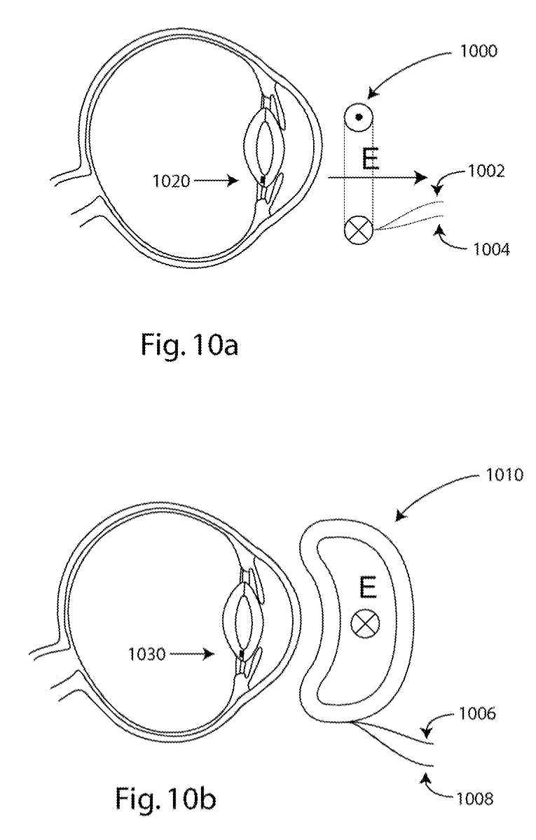

FIG. 9 shows the placement of a pressure sensor 900 incorporating the invention in the eye on an IOL haptic. Key features of the figure are the iris 910, an IOL 920, the lens capsule 930, the cornea 950 and a second IOL haptic 940. The pressure sensor can also be imbedded in the periphery of the IOL or attached to the tissues of the eye (not shown), including the iris 910. However, it is preferably placed outside of the optical path to the retina 960.

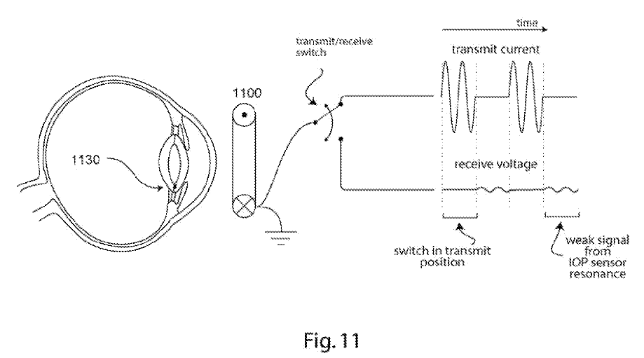

FIGS. 10a and 10b show possible placements of external coils 1000 and 1010 to interact with the magnetic material in the resonant structures of pressure sensors 1020 and 1030. FIG. 10a shows a geometry wherein a magnetic field is produced that is largely aligned with the optical path into the eye. The coil terminals are 1002 and 1004. FIG. 10b shows a geometry producing a field largely perpendicular to the optical path at the location of the sensor. The coil terminals are 1006 and 1008.

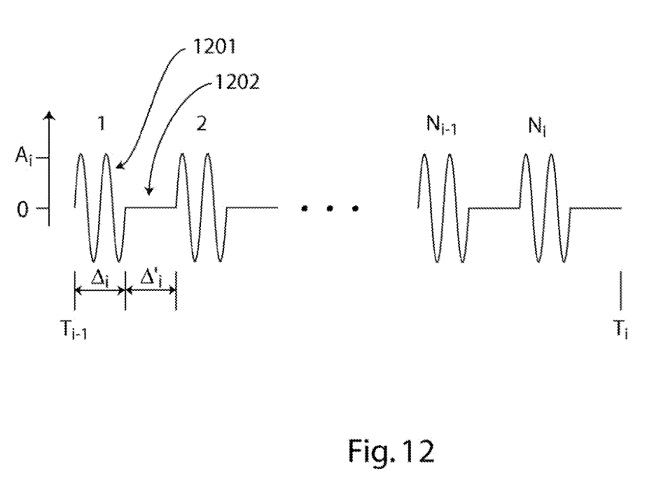

FIG. 11 depicts a signaling approach for communication with the pressure sensor. In particular, it depicts a sensor 1130 incorporating a resonant structure with an attached permanent magnet. The coil current is driven with pulsed tones. In between pulses, the coil 1100 is used to sense the oscillating magnetic field of the magnetic material. In this way, the high amplitude of the transmit signal does not interfere with the relatively weak signal produced by the vibrating magnet. The coil is alternately connected to the transmit circuitry and then to the receive circuitry with the analog transmit/receive switch as shown. The frequency of the pulsed tones is varied in order to search for a resonant frequency, or frequencies, of the sensor. This search is typically a coarse search to find the rough value of the frequencies and then fine searches to obtain accurate measurements of pressure. A useful feature of the signaling approach is the use of an analog switch to connect and disconnect the receive circuitry from the coil. Such an approach is referred to as a gated receiver. Although not shown, it should be understood that separate receive and transmit coils may be provided instead of the switched configuration discussed herein without straying from the intended scope of the present invention.

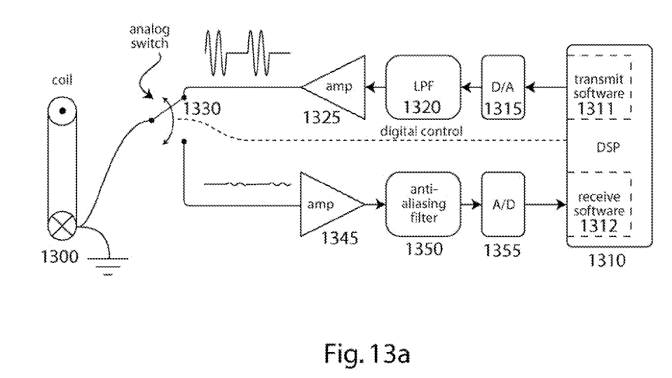

FIG. 12 describes in some detail the structure of a possible transmit current comprised of pulses (e.g. 1201) and quiet periods (1202). In order to detect a resonance at frequency fi, a total of Ni≧1 pulses of length Δi are transmitted with intervening quiet periods of a possibly different length, Δ′i. Switching distortion due to finite switching speed can be minimized by choosing Δi to be an integer multiple of sine wave periods corresponding to the test frequency fi. The intervening quiet periods are used by a receiver subsystem to detect weak signals produced by the oscillating permanent magnet on the resonant structure. This signal takes the form of a periodically modulated sine wave and hence contains sidebands in the frequency domain in addition to a large component at the frequency fi. To avoid having the side bands excite resonances, Ai can be chosen sufficiently short so that the sideband is out of the frequency range of interest. Alternatively, the sideband effects can be interpreted by the receiver, or the transmit current can be modulated, to spread the energy in the sidebands. The advantageous features of this transmit signal is that it has a significant spectral component at fi and periods of zero output where the receiver can detect varying magnetic fields emanating from the resonant structure. Systems incorporating such signals having quiet periods are referred to herein as having pulsed drive signals.

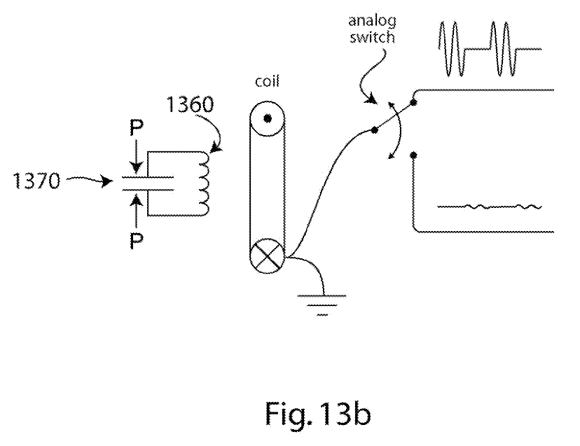

FIG. 13a shows a signal processing system (SPS) incorporating a digital signal processor (DSP) 1310. The DSP “transmit software” produces a digital version of the pulsed signal (or equivalent) depicted in FIG. 12. This signal is converted to an analog signal with a digital-to-analog converter (D/A) 1315, filtered by a low-pass filter (LPF) 1320 to remove effects of time sampling and then processed by an amplifier (amp) 1325. The resulting current signal is transmitted to a coil 1300 when the analog switch 1330 in the “up” position. In between pulses, the switch is in the “down” position. Magnetic signals from the resonant structure are communicated with the DSP via an amp 1345, an anti-aliasing filter 1350, and an analog-to-digital converter (A/D) 1355. The single electromagnetic coil can also be replaced with separate transmit and receive electromagnetic coils. Alternative approaches to signal processing involve continuous coil impedance measurements using a grid dip meter or equivalent. There are numerous ways of implementing the signal processing system so long as there is an excitation of the resonant structure and it interprets the vibrational motion of the resonant structure to estimate at least one resonant frequency and/or a sensed quantity.

FIG. 13b shows the electromagnetic coil attached to the signal processing system (SPS) interacting with an LC-type pressure sensor. In this embodiment a pressure-dependent capacitance 1370 is connected in parallel with a fixed inductor 1360 so that the resonant frequency of the LC circuit is pressure-dependent. The inductor is coupled magnetically to the coil portion of the signal processing system. Other LC sensors can be used in conjunction with the SPS so long as the sensed quantity causes variations in the capacitance and/or the inductance. The low signal-to-noise ratio problems associated with the low Q of LC resonators can be partially overcome with the SPS.

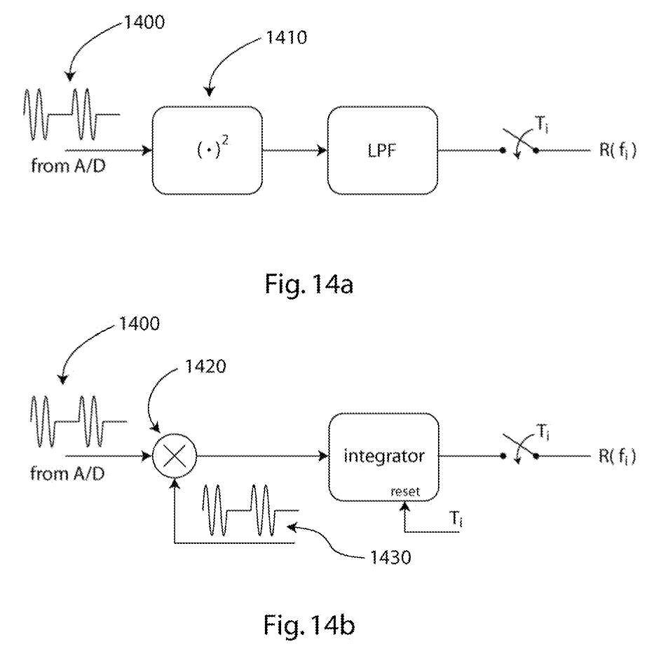

FIGS. 14a and 14b depict two block diagrams for the receiver software represented inside the DSP in FIG. 13. In general terms, the software is searching for the frequency(s) where the receiver gets a large response from the coil(s) near the sensor. The receive signal is represented by 1400 in FIGS. 14a and 14b. A simple processing technique is depicted in FIG. 14a and involves rectification (conversion to DC) using a squaring function 1410 followed by a low-pass filter (LPF). The LPF output is sampled at the end of the fi pulse train to create the response at this frequency denoted R(fi). Because this response depends on the signal amplitude and length of the pulse train, some normalization may be required. The rectification is shown with a squaring circuit, but other functions work as well, including an absolute value function and a time-synchronized demodulator which switches at the zero crossings. FIG. 14b shows the so-called matched filter approach to signal processing. The amplified receive signal is multiplied 1420 with the expected receive signal 1430 and integrated. At the end of the pulse train, at time T1, the integrated response is sampled to form R(fi) and the integrator is reset.

What is claimed and desired to be secured by Letters Patent is:

1. A sensing apparatus for measuring quantities convertible from changes in physical observations, said apparatus comprising:
a resonant structure responsive to said changes in said physical observations, said resonant structure including a magnetized element;
an electromagnetic coil operationally coupled to said magnetized element, said electromagnetic coil being an excitation coil magnetically coupled to said magnetized element to excite a resonance of said resonant structure; and,
a signal processor for processing movement of said resonant structure, said signal processor correlating said movement with regard to said changes in said physical observations so as to produce sensed data.

2. The apparatus as claimed in claim 1 wherein said changes in physical observations are changes in mechanical stress.

3. The apparatus as claimed in claim 1 wherein said changes in physical observations are changes in mass.

4. The apparatus as claimed in claim 1 wherein said sensed data includes physiological changes within a human body.

5. The apparatus as claimed in claim 4 wherein said physiological changes include changes in intraocular pressure.

6. The apparatus as claimed in claim 2 wherein said sensed data includes measurable physical occurrences selected from a group consisting of pressure changes, temperature changes, flow changes, rotation changes, acceleration changes, and sound changes.

7. The apparatus as claimed in claim 3 wherein said sensed data includes a measurable physical occurrence indicative of a presence of a chemical substance.

8. The apparatus as claimed in claim 2 wherein said resonant structure includes an adsorption mechanism that adsorbs a chemical substance such that said changes in physical observations is correlated to adsorption of said chemical substance by said adsorption mechanism.

9. The apparatus as claimed in claim 1 wherein said resonant structure resides within a vacuum environment so as to minimize damping losses.

10. The apparatus as claimed in claim 1 wherein said signal processor operates within a resonant sensing mode that is angular.

11. The apparatus as claimed in claim 1 wherein said signal processor operates within a resonant sensing mode that is linear.

12. The apparatus as claimed in claim 1 wherein said electromagnetic coil is also a pickup coil magnetically coupled to said magnetized element to sense a resonance of said resonant structure and to provide said resonance to said signal processor.

13. The apparatus as claimed in claim 1 wherein said electromagnetic coil is alternatively activated by circuitry within said signal processor to selectively form both said excitation coil and a pickup coil magnetically coupled to said magnetized element to sense said resonance of said resonant structure and to provide said resonance to said signal processor.

14. The apparatus as claimed in claim 1 wherein said resonant structure includes:
a substrate locatable in an environment to be monitored,
a flexible diaphragm hermetically sealed to said substrate and in communication with said environment to be monitored,
a sealed chamber encompassed by said substrate and said at least one flexible diaphragm, and
a resonant beam connected to said magnetized element, said resonant beam suspended within said sealed chamber and mechanically coupled to said flexible diaphragm,
wherein said magnetized element oscillates said resonant beam in response to an electromagnetic signal generated by said signal processor and formed by said electromagnetic coil.

15. The apparatus as claimed in claim 14 wherein said electromagnetic coil and said signal processor are locatable external to said environment to be monitored.

16. The apparatus as claimed in claim 15 wherein said environment to be monitored is intracorporeal, said substrate is attachable to a physiological structure, and said flexible diaphragm is capable of communication with a physiological fluid.

17. The apparatus as claimed in claim 16 wherein said substrate is attachable to a prosthetic device.

18. The apparatus as claimed in claim 16 wherein said environment to be monitored is an intraocular environment and said sensed data is intraocular pressure.

19. The apparatus as claimed in claim 17 wherein said environment to be monitored is an intraocular environment, said sensed data is intraocular pressure, and said prosthetic device is an intraocular lens.

20. The apparatus as claimed in claim 14 wherein said resonant beam is manufactured by photolithography and etching.

21. The apparatus as claimed in claim 14 wherein said substrate is formed from single crystal silicon.

22. The apparatus as claimed in claim 14 wherein said resonant beam is a polysilicon beam mounted to said substrate by at least one end of said polysilicon beam and spaced from said substrate between said at least once end and an opposite end of said polysilicon beam so as to allow free vibration of said polysilicon beam.

23. The apparatus as claimed in claim 22 wherein said polysilicon beam is formed from substantially undoped polysilicon treated to exhibit reduced tensile strain.

24. The apparatus as claimed in claim 14 wherein said flexible diaphragm is formed from polysilicon and surrounds said resonant beam, said flexible diaphragm being affixed to said substrate to define a primary cavity enclosing said resonant beam, said primary cavity being sealed off from surrounding atmosphere, and wherein an interior of said primary cavity is substantially evacuated.

25. The apparatus as claimed in claim 24 wherein said flexible diaphragm includes peripheral portions bonded to said substrate with channels extending through said peripheral portions from said primary cavity to a perimeter of said flexible diaphragm, said flexible diaphragm formed from material selected from a group consisting of silicon dioxide, polysilicon, silicon nitride, and combinations thereof, said material being formed within said channels and sealing off said channels such that atmospheric gases are prevented from entering or exiting said primary cavity through said channels.

26. The apparatus as claimed in claim 14 wherein said substrate further includes a displacement cavity, said displacement cavity sized such that a total internal cavity volume varies minimally with deflection of said flexible diaphragm over an operational range of displacement of said flexible diaphragm.

27. The apparatus as claimed in claim 14 wherein said resonant beam is suspended by said flexible diaphragm at one or more points thereupon such that said resonant beam is suspended beneath said flexible diaphragm.

28. The apparatus as claimed in claim 24 further including a depression in said substrate forming said primary cavity, wherein said resonant beam is attached to said flexible diaphragm in at least one point and to said substrate in at least another point.

29. The apparatus as claimed in claim 24 wherein said resonant beam is attached to said flexible diaphragm in at least two points such that said resonant beam is suspended entirely by said flexible diaphragm.

30. The apparatus as claimed in claim 14 wherein said resonant beam includes a stress-sensitive coating affixed thereon for varying stiffness of said resonant beam such that said resonant beam exhibits a variable resonant amplitude.

31. The apparatus as claimed in claim 14 wherein said resonant beam forms a structure selected from a group consisting of a bridge, a double ended tuning fork (DEFT), a cantilever, and a diaphragm.

32. The apparatus as claimed in claim 14 wherein said resonant beam is dynamically balanced.

33. The apparatus as claimed in claim 14 wherein said resonant beam exhibits mechanical amplification.

34. The apparatus as claimed in claim 14 wherein said resonant beam includes two resonant structures that are each used in a differential mode.

35. The apparatus as claimed in claim 14 wherein said magnetized element is formed from a permanent magnet.

36. The apparatus as claimed in claim 14 wherein said magnetized element is formed from a soft magnetic material.

37. The apparatus as claimed in claim 14 wherein said magnetized element is electroplated onto said resonant beam.

38. The apparatus as claimed in claim 14 wherein said magnetized element is formed from a conductor loop that exhibits a magnetic field in response to said electromagnetic signal.

39. The apparatus as claimed in claim 14 wherein said signal processor includes at least one gated receiver.

40. The apparatus as claimed in claim 14 wherein said signal processor forms at least one pulsed drive signal.

41. The apparatus as claimed in claim 14 wherein said signal processor is a grid dip meter.

42. The apparatus as claimed in claim 14 wherein motion of said resonant beam is detected optically.

43. The apparatus as claimed in claim 14 wherein motion of said resonant beam is detected acoustically.

44. The apparatus as claimed in claim 14 wherein motion of said resonant beam is detected electromagnetically by way of said electromagnetic coil in operational coupling with said signal processor.

45. A method of sensing physical observations within an environment, said method comprising:
operatively arranging a resonant structure in said environment and in proximity to a direct current bias field, said resonant structure including a magnetized element and being responsive to changes in said physical observations;
applying a magnetic field by way of an electromagnetic coil operationally coupled to said magnetized element;
measuring a plurality of successive values for magnetic resonance intensity of said resonant structure with a signal processor operating over a range of successive interrogation frequencies to identify a resonant frequency value of said resonant structure; and
using said resonant frequency value to identify sensed data correlating to said physical observation of said environment.

46. The method as claimed in claim 45 wherein said magnetic field is a time-varying magnetic field.

47. The method as claimed in claim 45 wherein said magnetic field is a magnetic field pulse.

48. The method as claimed in claim 45 wherein said magnetic field is a series of magnetic field pulses.

49. The method as claimed in claim 45 wherein said electromagnetic coil is an excitation coil magnetically coupled to said magnetized element to excite a resonance of said resonant structure.

50. The method as claimed in claim 49 wherein said signal processor processes movement of said resonant structure and correlates said movement with regard to said changes in said physical observations so as to produce said sensed data.

51. The method as claimed in claim 45 further including a step of detecting a transitory time-response of frequency emission intensity of said resonant structure with a receiver to identify a resonant frequency value of said resonant structure to be used for determining said sensed data.

52. The method as claimed in claim 51 further including a step of converting said detected transitory time-response into a frequency domain format so as to enable performance of a Fourier transform on said transitory time-response of magnetic vibration intensity detected.

53. The method as claimed in claim 45 further including steps of providing soft magnetic material exterior to said resonant structure, so as to increase signal detection by said signal processor.

54. An apparatus for measuring quantities convertible from changes in physical observations, said apparatus comprising:
a resonant structure responsive to said changes in said physical observations, said resonant structure including a magnetized element;
an electromagnetic coil operationally coupled to said magnetized element, said electromagnetic coil being magnetically coupled to said magnetized element; and,
a signal processor for processing movement of said resonant structure, said signal processor correlating said movement with regard to said changes in said physical observations so as to produce sensed data.

55. The apparatus as claimed in claim 54 wherein said electromagnetic coil is a pickup coil magnetically coupled to said magnetized element to sense a resonance of said resonant structure and to provide said resonance to said signal processor.

56. The apparatus as claimed in claim 54 wherein said electromagnetic coil is an excitation coil magnetically coupled to said magnetized element to excite a resonance of said resonant structure.

57. The apparatus as claimed in claim 54 wherein said electromagnetic coil is alternatively activated by circuitry within said signal processor to selectively form both an excitation coil and a pickup coil magnetically coupled to said magnetized element to sense said resonance of said resonant structure and to provide said resonance to said signal processor.

58. The apparatus as claimed in claim 54 wherein said resonant structure is a resonant LC circuit.

59. The apparatus as claimed in claim 58 wherein said signal processor includes at least one gated receiver.

60. The apparatus as claimed in claim 58 wherein said signal processor forms at least one pulsed drive signal.

61. The apparatus as claimed in claim 54 further including more than one resonant structure, each said resonant structure responsive to differing ones of said physical observations.

62. The apparatus as claimed in claim 54 further including more than one electromagnetic coil, at least one of said more than one electromagnetic coils being a pick up coil magnetically coupled to said magnetized element to sense a resonance of said resonant structure and to provide said resonance to said signal processor, and at least another of said more than one electromagnetic coils being an excitation coil magnetically coupled to said magnetized element to excite a resonance of said resonant structure.
